(19) United States
(12) Patent Application Publication
(10) Pub. No.: US 2007/0021706 A1
(43) Pub. Date: Jan. 25, 2007

(54) Orthopedic devices with compressive elastomer formed directly onto a base material

(75) Inventors: Sam Braunstein, New York, NY (US); Jeffrey Stearns, Hopatcong, NJ (US)

Correspondence Address: WALL MARJAMA & BILINSKI, 101 SOUTH SALINA STREET, SUITE 400, SYRACUSE, NY, 13202, US

(73) Assignee: Wellgate Products, LLC, New York, NY (US)

(21) Appl. No.: 11/185,333

(22) Filed: Jul. 20, 2005

(51) Int. Cl. A61F 13/06; A61F 13/00

(52) U.S. Cl. 602/63; 602/62

(57) ABSTRACT

Orthopedic support devices are provided that include a first layer of elastomeric material is attached directly atop or around a layer of a spacer material, wherein the first layer provides support and/or compression, and the second layer provides breathability and wicking and wherein characteristics of the first layer can be varied in order to vary the location, amount and/or level of compression provided by the support device.

### FIELD OF THE INVENTION

The present invention relates to orthopedic devices, and, in particular, to orthopedic support devices that comprise an elastomeric material formed directly onto, atop, or around a base material in order to provide not only support but also a desirable combination of compression, protection and suspension to an injured body part.

### BACKGROUND OF THE INVENTION

Many people develop injuries in an area of their body (e.g., knee, ankle, elbow, wrist) that is utilized on a daily basis such that the injured area cannot be immobilized while the injury heals. Thus, the goal becomes to stabilize and protect the injured body part to an extent whereby some usage of the body part can occur while still allowing for there to be simultaneous healing. To that end, orthopedic support devices have been developed consisting of a layer of flexible, resilient material (e.g., neoprene) which, when stretched over a body part, provides support thereto.

Various problems have been observed with regard to these traditional orthopedic devices. For example, resilient materials neither effectively dissipate heat nor absorb/wick perspiration away from the skin. Thus, those who wear devices formed of such materials in warm climates and/or while engaged in strenuous physical activity may develop skin irritation, abrasions, heat rashes and/or dermatitis due to perspiration, particularly at points of bending such as the back, the knee, the elbow or the wrist.

Moreover, conventional resilient material orthopedic supports tend to migrate from their desired area of coverage, again owing to perspiration. Migration leaves the injured area entirely or partially unsupported, which, in turn, can result in slowed healing or even aggravation of the underlying injury. In a similar vein, resilient orthopedic supports have been known to sag, lose its shape or “bunch up,” e.g., when the supported body part is flexed. Bunching also can leave injured areas unprotected or only partially protected, and can either expedite the onset of skin problems already associated with such devices or create still other skin problems such as chafing or bruising.

Those in the art have made various attempts to solve these problems, including by providing support devices with multiple layers. However, these multilayer supports tend to be comparatively bulkier and/or more expensive than single layer supports. Plus, the presence of the extra layer(s) tend to cause discomfort to the wearer of the device, and/or may exacerbate skin problems caused by single layer devices.

One particular manufacturing approach that seeks to curb the problem of “bunching” is to utilize compression molding to form the support device. However, this compression molding technique tends to produce a support that, although bunching-resistant, lacks the flexibility and elasticity to be worn comfortably and to permit an acceptable range of movement for the injured area.

To combat the problem of support devices sagging or losing their shape, some have produced devices that include a buttress made of a comparatively stiffer material. However, the added firmness of the buttress can irritate the injured area if the buttress is either too firm and/or placed too near the injured area. Moreover, the process for attaching the buttress (e.g., sewing, gluing) is labor-intensive and the end product may not be the ideal shape and size for people of different anatomies. Customization is an option; however, that renders the manufacturing process slower and more expensive for only mildly improved results.

There have been other attempts to stiffen orthopedic supports without the addition of a buttress, such as through the use of an injection molding manufacturing process. However, support devices produced by injection molding suffer from similar drawbacks as those that include buttresses, e.g., limited range of motion and comparative lack of compression. Moreover, injection molding equipment is particularly expensive to implement and operate.

Two other approaches have likewise proven unsuccessful, namely the insertion of webbing within cuts in the support material and utilization of standalone gel components that are attached by straps or other devices.

Therefore, a need exists for orthopedic supports that avoid the litany of problems of conventional devices, yet that still can promote healing and enable freedom of movement while being worn.

### SUMMARY OF THE INVENTION

These and other needs are met by the present invention, which, according to an exemplary aspect of the invention, provides orthopedic support devices that include a support body wherein a first layer formed of first material (e.g., an elastomeric material) is attached directly atop or around a second layer formed of a different material (e.g., a spacer material), wherein the first material layer provides support and/or compression, and the second material layer provides breathability and wicking.

The present invention advantageously provides flexibility with regard to both design and treatment options for orthopedic support devices, since certain characteristics (e.g., hardness, shape, thickness, location, pattern, modulus of elasticity) of the first material layer can be varied precisely in order to controllably vary the location and/or the amount/level of compression provided by the support device. That, in turn, enables orthopedic support devices in accordance with the present invention to provide targeted compression in order to offer an optimal combination of healing, comfort and freedom of movement while the devices are being worn, yet without resulting in devices that are prohibitively expensive to produce.

By way of non-limiting example, the support body for the orthopedic support device can be a sleeve wherein the first material layer is formed to have a constant pattern (e.g., a web-like pattern with openings defined therein) and a substantially constant thickness to provides a substantially constant level/amount of compressive force to the injured body part (e.g., a knee) around which the sleeve is worn. To provide varied compression to the injured body part, the thickness of the first layer can be modified (e.g., increased by providing a buttress) in certain predetermined areas of the sleeve and/or the constant pattern can be made non-uniform.

According to another exemplary aspect of the present invention, the support body for an orthopedic sleeve includes elastomeric material in the form of one or more buttresses that are sited on the sleeve such that when the sleeve is worn the buttress(es) correspond to the location of an injured area and thus provide highly targeted compression thereto. By way of non-limiting example, an orthopedic sleeve meant to be worn over a knee can include an elastomeric buttress that, when the sleeve is properly worn, will be positioned over the kneecap and/or a knee ligament.

In accordance with yet another exemplary aspect of the present invention, the support body for the orthopedic support device can be in the form of a support strap, e.g., a patella tendon bearing strap, whereby certain portions of the strap can be formed either entirely or partially of the elastomeric material in order to provide targeted compression to certain injured areas, e.g., the patella tendon.

Still other aspects, embodiments and advantages of the present invention are discussed in detail below.

### BRIEF DESCRIPTION OF THE DRAWINGS

For a fuller understanding of the nature and desired objects of the present invention, reference is made to the following detailed description taken in conjunction with the accompanying figures, wherein like reference characters denote corresponding parts throughout the views, and in which:

FIG. 1 is front, perspective view of an orthopedic support sleeve of the present invention being worn on a knee;

FIG. 2 is a top view of the orthopedic support sleeve of FIG. 1 in an unworn condition;

FIG. 3 is a front, perspective view of an alternative embodiment of the orthopedic support sleeve of FIG. 1 being worn on a knee;

FIG. 4 is top view of a front panel of an orthopedic support sleeve of the present invention that includes a shaped buttress;

FIG. 5 is a side, sectional view of the front panel of FIG. 4 along the line 5-5;

FIG. 6 is an exploded view of the front panel of FIG. 4;

FIG. 7 is a top view of orthopedic patellar tendon bearing support device of the present invention;

FIG. 8 is a top view of the support buttress of the orthopedic device of FIG. 7

FIG. 9 is a bottom view of the support buttress of the orthopedic device of FIG. 7; and

FIG. 10 is a front view of the orthopedic support device of FIG. 7 as worn.

### DETAILED DESCRIPTION

The present invention provides embodiments of orthopedic support devices in which a first material layer is attached directly onto, atop, or around a second material layer, wherein the first material layer provides support and/or compression, and the second material layer provides breathability and/or wicking. As will be described in detail below, and in accordance with such embodiments, certain characteristics (e.g., hardness, modulus of elasticity, shape, thickness, and/or location) of the first material layer can be varied to vary the location and/or the amount/level of compression provided by the support device. That, in turn, enables cost effective formation of an support device that can provide targeted compression to an injured body part with an optimal combination of healing and freedom of movement while the device is being worn.

In such embodiments, it is the first material layer that generally provides compression when the orthopedic device is worn. In an exemplary embodiment of the present invention, the first material layer is formed of one or more elastomeric or rubber materials. Suitable such materials include, but are not limited to materials having a hardness in the range of Shore 00-30 to Shore A-50 and/or a modulus of elasticity in the range of about 20 psi to about 150 psi, such as a thermoplastic rubber material (e.g., a thermoplastic elastomer), a silicone material (e.g., a silicone elastomer), a polyurethane material, a polyvinylchloride material, a styrene material (e.g., styrene-butadiene rubber, styrene-butadiene rubber and natural rubber blend), an acrylic rubber (e.g., polyacrylate, ethylene acrylic rubber), a polyester-urethane (e.g., ADIPRENE®), a butadiene-based material (e.g., polybutadiene, butyl rubber, hi-nitrile butadiene rubber), a choloroprene material (e.g., neoprene), a chlorosulfonated elastomer, an ethylene copolymer, an ethylene vinyl acetate material, a fluoro-rubber, a natural rubber, a nitrile elastomer, a hi-nitrile phenolic rubber, an epicholorohydrin material, and a vinyl plasticol material.

Generally, the material that forms the first material layer will have a higher modulus of elasticity than the material that forms the second material layer, so as to provide the orthopedic support with a desirable overall combination of support, compression and breathability, while still allowing for freedom of movement of the area on or over which the support is worn. However, it is understood that the material that forms the first material layer can have a modulus of elasticity less than or equal to the modulus of elasticity of the material that forms the second material layer without departing from the scope of the present invention.

The second material layer generally provides a buffer layer between the first material layer and the wearer's skin, but can have other functions and placements as well. According to an exemplary embodiment of the present invention, the second material layer is made of a material that is substantially breathable, has good wicking characteristics, and/or is resistant to bunching and migration when the orthopedic device is worn. Suitable such materials include, but are not limited to elastic or inelastic spacer materials such as nylon or polyester.

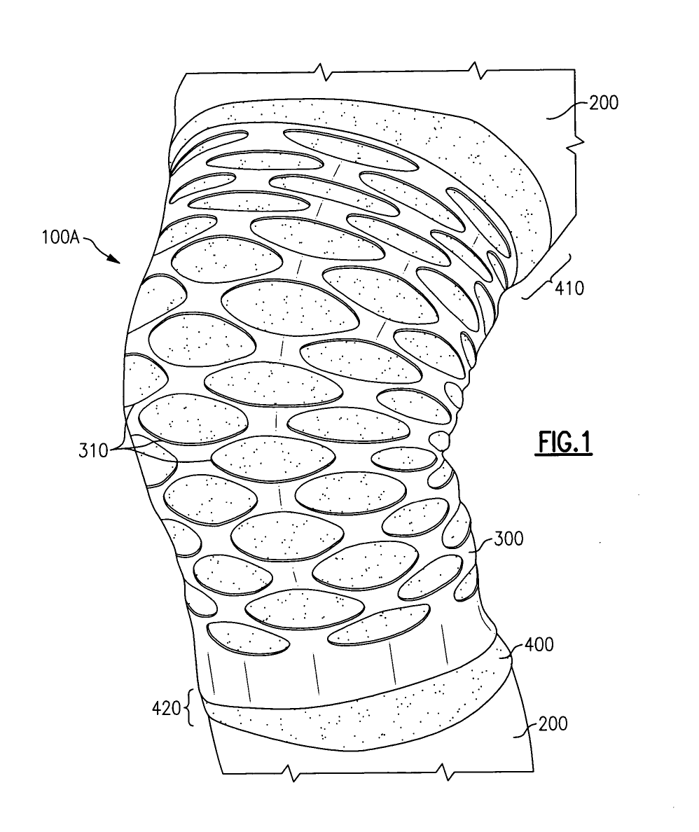

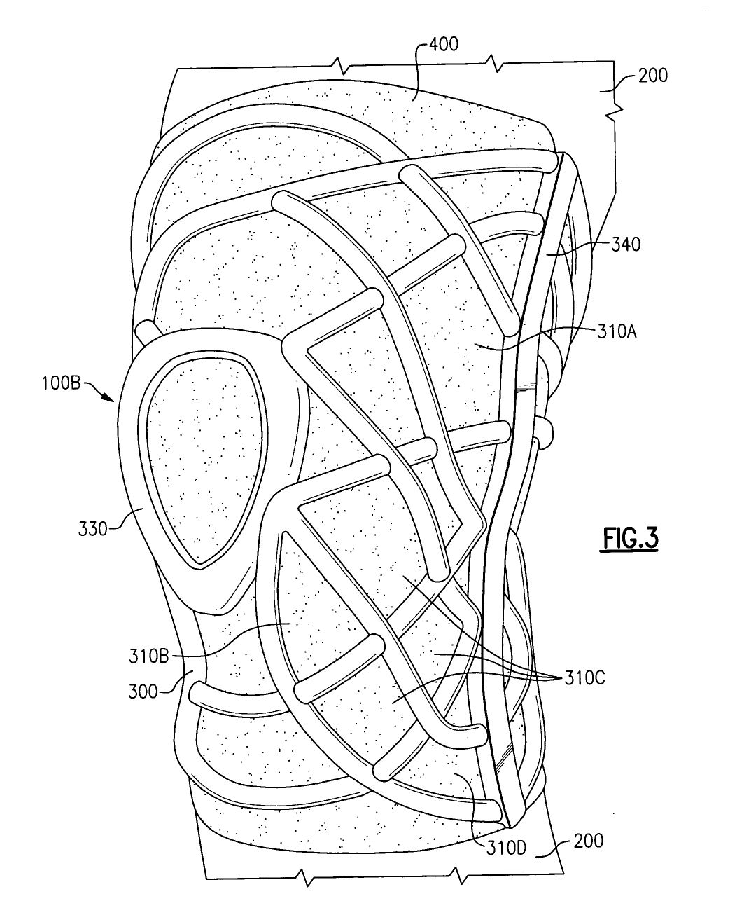

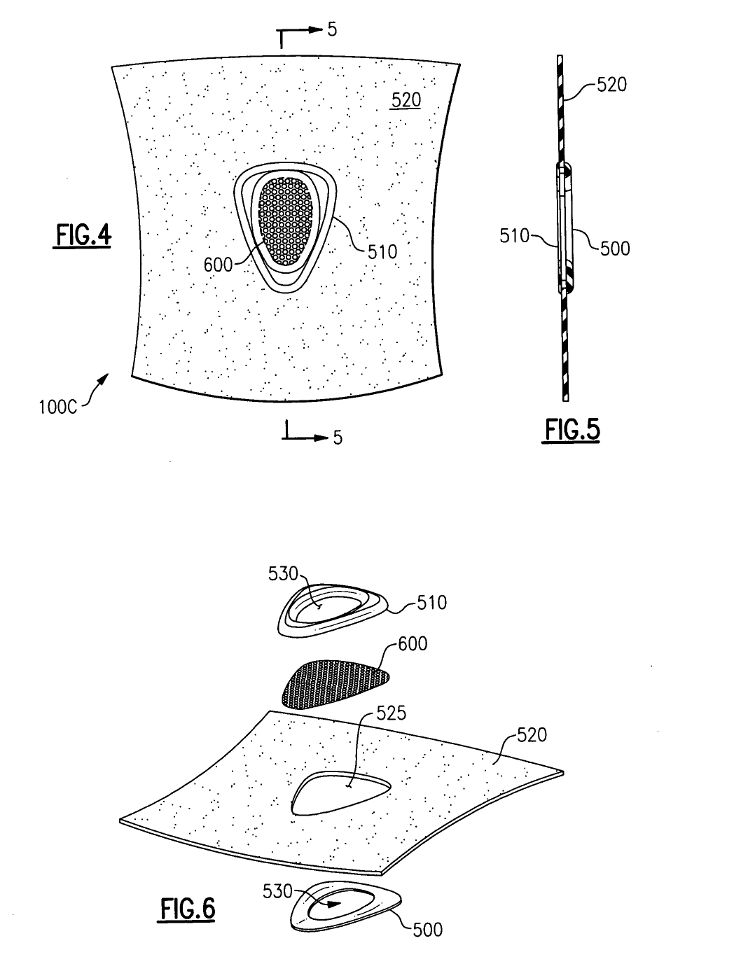

In accordance with various exemplary embodiments of the present invention, the first material layer can have a substantially constant or varied thickness, design and/or shape over some or all of its overall length. For example, and as will be discussed in greater detail below, the first material layer can have a uniform patterned shape of substantially constant thickness (see, e.g., the sleeve 100A illustrated in FIG. 1) or the first material layer can have a non-uniform patterned shape of non-constant thickness (see, e.g., the sleeve 100B illustrated in FIG. 3), and/or the first material layer can entirely comprise a shaped area of added thickness (see, e.g., the sleeve 100C depicted in FIGS. 4-6).

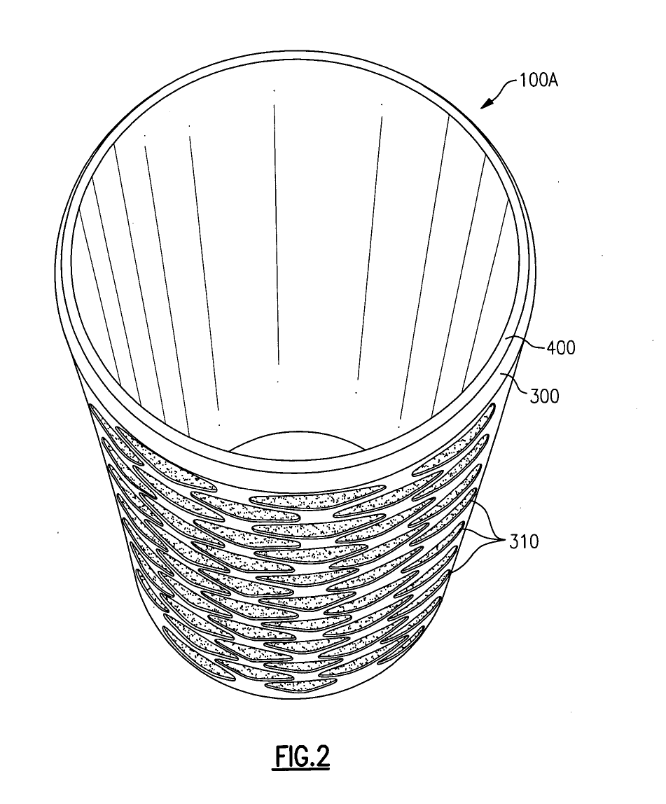

Referring initially to FIGS. 1 and 2, an exemplary embodiment of an orthopedic support device 100A according to the present invention is shown. In the depicted embodiment, the support device 100A is a sleeve, which, as shown in FIG. 1, can be worn over a portion of a wearer's leg 200 such that the first material layer 300 provides a compressive force on and around the wearer's knee and such that the second material layer 400 is in direct contact with the wearer's skin, thus acting as a barrier layer between the first material layer and the wearer.

The overall length of the second material layer 400 of the sleeve 100A can be less than, substantially equal to or greater than the overall length of the first material layer 300 of the sleeve 100A. As depicted in FIG. 1, and in accordance with an exemplary embodiment of the present invention, the length of the second material layer 400 of the sleeve 100A is greater than the length of the first material layer 300 such that one or more portions 410, 420 of the second material layer 400 extend above and/or below the first material layer 300, thus enabling the sleeve 100A to serve as a lower modulus control top in order to minimize displacement of soft tissue.

According to an alternate embodiment of the present invention, the extended portion(s) 410 and/or 420 can be formed of a third material different from the material that comprises either the first or second material layers 300, 400. By way of non-limiting example, and in accordance with such an alternate embodiment, the first material layer 300 can be formed of an elastomeric layer, the second material layer 400 can be formed of a spacer material, and the third material that forms the extended portion(s) 410 and/or 420 can be formed of a resilient material (e.g., neoprene).

As best illustrated in FIG. 2, the first and second material layers 300, 400 are present around the entire circumference of the sleeve 100A in accordance with this exemplary embodiment of the present invention. FIG. 2 also illustrates that the first material layer 300 is formed directly atop/around and not encapsulated within the second material layer 400; however, it is understood that can be at least partially encapsulated within the second material layer 400 if instead desired. Also, although FIGS. 1 and 2 depict the first material layer 300 being present around the entire sleeve 100A (i.e., having 360° of coverage), it should be noted that the amount of coverage of the first material layer 300 can be about less than 360° in accordance with the present invention, e.g., such that when the sleeve device 100A is worn the first material layer can correspond to the front, back or side of a knee.

FIGS. 1 and 2 depict an exemplary embodiment of a sleeve 100A wherein the first material layer 300 has a substantially repeating, patterned shape, which, in this exemplary embodiment, resembles a web, but which can take on other shapes as well. To form the FIG. 1 pattern, the first material layer 300 of the sleeve 100A has a plurality of linked circular or elliptical openings 310 defined therein. The presence of these openings 310 is beneficial, since they better enable dissipation of heat and/or moisture (i.e., heat and/or moisture that has accumulated between the wearer's skin and the second material 400) through the breathable second material layer 400, thus reducing the occurrence, or at least the severity of skin problems associated with heat and/or moisture retention between the sleeve 100A and the wearer's skin. Moreover, the web-like pattern of openings 310 within the first material layer 300 provides an improved fit as well as increased comfort and an increased range of motion to the wearer, while still providing necessary targeted compression along substantially the entire sleeve 100A.

In an exemplary embodiment of the present invention wherein the sleeve 100A is designed for placement over a knee, the diameter of each opening 310 is generally in the range of about 0.25 inch to about 1.25 inch, and the total number of openings is generally in the range of about 100 about 200, and the distance between each opening is generally in the range of about 0.15 inch to about 0.80 inch. It is understood that the number, and/or the diameter, and /or the distance between one, some or all of the openings 310 within the first material layer of sleeve 100A can be modified within or outside these ranges for various reasons, e.g., to modify the level of compression sought to be provided by the sleeve 100A, to provide non-uniform compression at one or more areas, to adapt to an atypical anatomy of a wearer, to relieve compression at one or more pressure points, and/or to vary the range of motion provided to a wearer, and/or to improve the overall fit of the device. It is further understood that one or more portions of the first material layer 300 between and/or around the openings 310 can have various complex and/or compound radii and/or can have filleted areas and/or edges, thus allowing the modulus of elasticity of such portion(s) to be precisely tailored.

In accordance with an exemplary embodiment of the present invention in which the first material layer 300 has a plurality of openings 310 defined therein, at least a portion of the length of the first material layer does not include any openings in order to enhance the structural integrity of the device. By way of non-limiting example, such a portion can be located at the bottom and/or top end(s) of the first material layer 300 in order to prevent migration and bunching and to increase the durability of the sleeve 100A. Also by way of non-limiting example, and as shown in FIG. 1, such a portion can be located at the bottom end 320 of the first material layer 300 and generally will have a length in the range of about 0.10 inch to about 2.50 inch, wherein this length can be modified within or outside of this range for various reasons, e.g., to improve the overall fit of the device.

Because of the presence of the repeating pattern of openings 310 within its first material layer 300, the sleeve 100A of FIG. 1 provides substantially uniform, targeted compression and pressure when worn over an injured body part. It may be desired, however, to provide varied (i.e., non-uniform) compression through some portions of a sleeve 100A. For example, it may be desired to design the sleeve 100A to provide increased compression at the portion of the sleeve that will be worn directly over an injured area, e.g., a kneecap or a knee ligament. Such varied compression can be accomplished, e.g., by varying one or more of the shape of the openings 310 and/or by varying the thickness of the first material layer 300, and/or by varying the modulus of elasticity of the first material layer.

Referring now to FIG. 3, an alternate embodiment of the FIG. 1 sleeve 100A is shown that differs from the FIG. 1 sleeve in that its sleeve 100B provides a non-uniform level of compression by including a varied pattern of openings 310 and a varied thickness of the first material layer 300 in certain predetermined areas of the sleeve. Whereas the pattern of the first material layer 300 in the FIG. 1 sleeve 100A was substantially repeating, the pattern of the first material layer in the FIG. 3 sleeve 100B is substantially non-uniform (i.e., non-repeating). By way of non-limiting example, and as shown in the FIG. 3 exemplary embodiment, some openings 310A in the first material layer 300 of the sleeve 100B can have a pentagonal shape, while other openings 310B can have a triangular shape, and still other openings 310C can have a rhomboid shape, and yet still other openings 310D can have a trapezoidal shape. Each of these shapes represents not only a differently shaped opening, but also a somewhat differently sized opening; thus, in turn, each differently shaped opening provides a somewhat different level of compression beneath the opening. In general, the number of total openings 310A, 310B, 310C, 310D and the distance between each opening provided in the FIG. 3 sleeve 100B will be less than the total number. of openings 310 and the distance between each opening in the FIG. 1 sleeve, whereas the size of the openings 310A, 310B, 310C, 310D in the FIG. 3 sleeve 100B generally will be greater than the size of the openings 310 in the FIG. 1 sleeve 100A.

Further, whereas the thickness of the first material layer 300 was substantially constant in the FIG. 1 sleeve 100A, one or more portions of the first material layer in the FIG. 3 sleeve 100B generally will have an increased thickness in order to provide one or more areas of increased, targeted compression. Also, the added thickness will prevent or at least further deter bunching and migration of the sleeve 100B. The number, location and/or shape of these increased thickness portions can vary; however, according to an exemplary embodiment of the present invention, the FIG. 3 sleeve includes at least two added thickness portions: a first added thickness portion 330 (i.e., a buttress) surrounding one of the circular openings 310 of the first material layer 300 that provides increased compression at a kneecap, and a second added thickness portion 340 that spans between the top and bottom ends of the first material layer to provide increased compression to the knee ligaments and to specifically deter migration and bunching of the sleeve 100B.

In the FIG. 1 embodiment 100A of the present invention, the thickness of the first material layer 300 is substantially constant and is generally in the range of about 0.05 inch to about 0.20 inch, and the thickness of the second material layer 400 is substantially constant and is generally in the range of about 0.05 inch to about 0.25 inch. In the FIG. 3 embodiment 100B, the thickness of the second material layer 400 is generally constant and within the same range as its thickness in the FIG. 1 embodiment and the thickness of the first material layer 300 is generally constant and within the same range as its thickness in the FIG. 1 embodiment, except at the increased thickness portions 330, 340 where the thickness of the first material layer is generally up to about 50% greater than that of the remainder of the first material layer. By way of non-limiting example, in a FIG. 3 embodiment wherein the thickness of the first material layer 300 is about 0.125 inch, the thickness of each of the increased thickness portions 330, 340 generally will be up to about 0.1875 inch. The hardness of the first material 300 generally will be about 20 Shore A in both the FIG. 1 embodiment 100A and the FIG. 2 embodiment 100B.

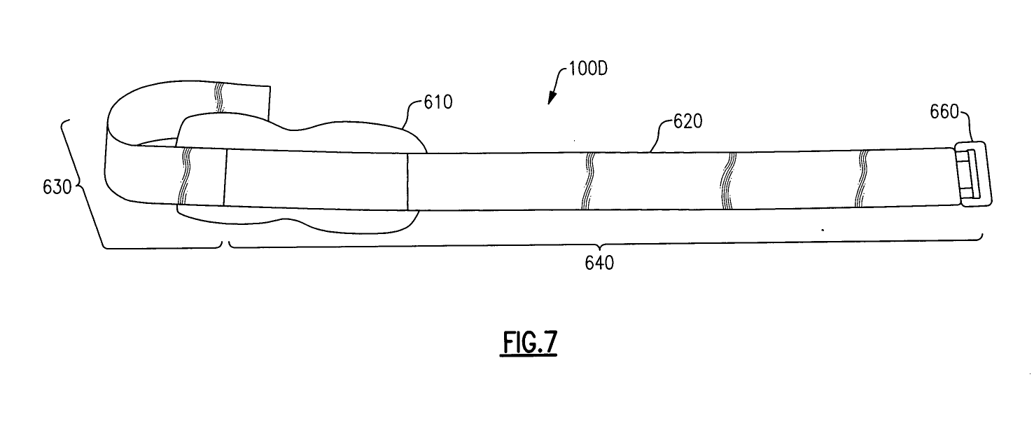

As noted above, the FIG. 3 sleeve 100B includes a buttress 330, which comprises a portion of the first material layer 300 and which has comparatively increased thickness to provide increased targeted compression. FIGS. 4-10 depict other exemplary embodiments of orthopedic devices 100C (see FIG. 4-6), 100D (see FIG. 7-10) in accordance with the present invention that also include one or more buttress portions formed at least partially of an elastomeric material.

FIGS. 4-6 depict a top panel of an orthopedic device 100C. The device 100C can be a sleeve that is worn over a knee, in which case the top panel can be connected to a back panel (not shown) or can be fashioned into a sleeve, e.g., by extending the front panel to form a sleeve that would fit over a leg as do the sleeves 100A, 100B shown in FIGS. 1-3. The device 100C includes a panel/sleeve body 520 having an opening 525 (see FIG. 6), wherein a first buttress 500 is formed on the inside of the panel body and a second buttress 510 is formed on the outside of the panel body. According to an exemplary embodiment of the present invention, the first and second buttresses 500, 510 are located at the same position on the device 100C, wherein that position will correspond to a kneecap when the device is worn over a leg, and wherein the first, inner buttress 500 would be in direct contact with the kneecap. Each buttress 500, 510 generally includes an opening 530, wherein at least a portion of each buttress opening will overlap at least a portion of (optionally, substantially the entirety of) the opening 525.

Although two buttresses 500, 510 are illustrated in FIGS. 4-6, it should be noted that the FIG. 4 device 100C can include only one buttress or greater than two buttresses. In an embodiment wherein the device 100C includes only one buttress, the buttress generally would be located on the inside of the panel body 520, e.g., in a position that will correspond to a kneecap when the device is worn over a leg. In an embodiment wherein more than two buttresses are included, the additional buttress(es) can be located, e.g., inside and/or outside the sleeve 100C so as to correspond to positions at either or both sides of the knee in order to protect knee ligaments.

The first and second buttresses 500, 510 can have the same or different shapes and the same or different thicknesses. According to the exemplary embodiment of the FIGS. 4-6 sleeve 100C, the first and second buttresses 500, 510 have identical “shieldlike” shapes (i.e., resembling a rounded triangle) and each buttress has an elliptical opening 530 defined therein. Without wishing to be bound by theory, such a shape is believed to be well suited for providing targeted support and compression to a kneecap when the sleeve 100C is worn over a knee; however, other shapes for the buttresses 500, 510 and/or the openings 530 are within the scope of the present invention. The size of the openings 530 can vary, as can the size of the buttresses 500, 510 in which the opening is defined; however, according to an exemplary embodiment of the present invention, each opening covers about 35% to about 80% of the overall surface area of each buttress.

According to an exemplary embodiment of the present invention, and as best shown in FIG. 6, a fabric layer 600 can be provided between the inner buttress 500 and the outer buttress 510. The fabric layer 600 is selected to be highly breathable to allow air and/or moisture generated within the openings 530 to be breathed and/or wicked away from the skin. According to an exemplary embodiment of the present invention, the buttresses 500, 510 are made of an elastomeric material, the panel body 520 is made of a resilient material (e.g., neoprene) and the fabric layer 600 is made of mesh material.

Optionally, the device 100C can include first and second control top portions 610, 620, which are provided at a top portion and a bottom portion of the sleeve/panel body 520 as described above with regard to the FIG. 1 sleeve 100A. According to an exemplary embodiment of the present invention, the control top portions 610, 620 of the device 100C are made of a spacer material and again are present in order to minimize displacement of soft tissue.

The thickness and/or the hardness of each buttress 500, 510 can be identical or can vary. According to an exemplary embodiment of the present invention, the thickness of the first, inner buttress 500 is greater than the thickness of the second, outer buttress 510 and the hardness of the first, inner buttress is less than the hardness of the second, outer buttress. This enables the first, inner buttress 500 to provide cushioned support and the second, outer buttress 510 provides firmer, structural support.

The first, inner buttress 500 is generally about three to five times (e.g., four times) as thick as the second, outer buttress 510, wherein the thickness of the first, inner buttress 500 generally is in the range of about 0.15 inch to about 0.35 inch (e.g., about 0.25 inch) and the thickness of the second, outer buttress 510 generally is in the range of about 0.0425 inch to about 0.0825 inch (e.g., about 0.0625 inch). The hardness of the first, inner buttress 500 generally is about 40 Shore 00, and the hardness of the second, outer buttress 510 generally is about 40 Shore A.

Another exemplary orthopedic support device 100D in accordance with the present invention is shown in FIGS. 7-10. This device 100D is patella tendon bearing device and includes a support buttress 610 having an attached strap 620, wherein the strap has a first strap portion 630 and a second strap portion 640, and wherein the second strap portion culminates in a D-ring or buckle 660 that is configured to accept the first strap portion 650. By way of non-limiting example, the first strap portion 630 can be made of hook material (e.g., velcro) and the second strap portion 640 can be made of loop material (e.g., velcro) to help maintain the device 100D in place when worn as shown in FIG. 10.

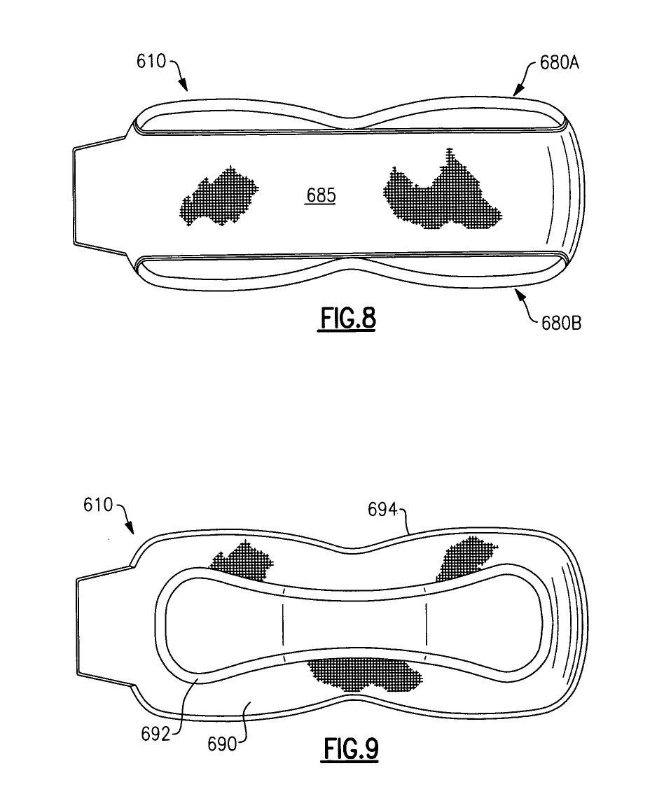

As best shown in FIG. 8, the top side 670 of the support buttress 610 includes first and second raised portions 680A, 680B (e.g., rails) shaped to function as a strap guide, wherein a strap guide area 685 is defined between the first and second rails on which the strap portion 620 will rest when the device 100D is being worn (see FIG. 10). In accordance with an exemplary embodiment of the present invention, the first and second raised potions 680A, 680B of the support buttress 610 are made solely of an elastomeric material, and the strap guide area 685 between the first and second raised portions are made of a combination of an elastomeric material and a spacer material. Generally, the same elastomeric material is utilized to form both raised portions 680A, 680B of the buttress 610, wherein according to an exemplary embodiment of the present invention the hardness of the elastomeric material is about 40 Shore A. The elastomeric material within the combination of materials utilized to form the strap guide area 685 can have the same or different hardness as the elastomeric material that comprises the raised portions 680A, 680B.

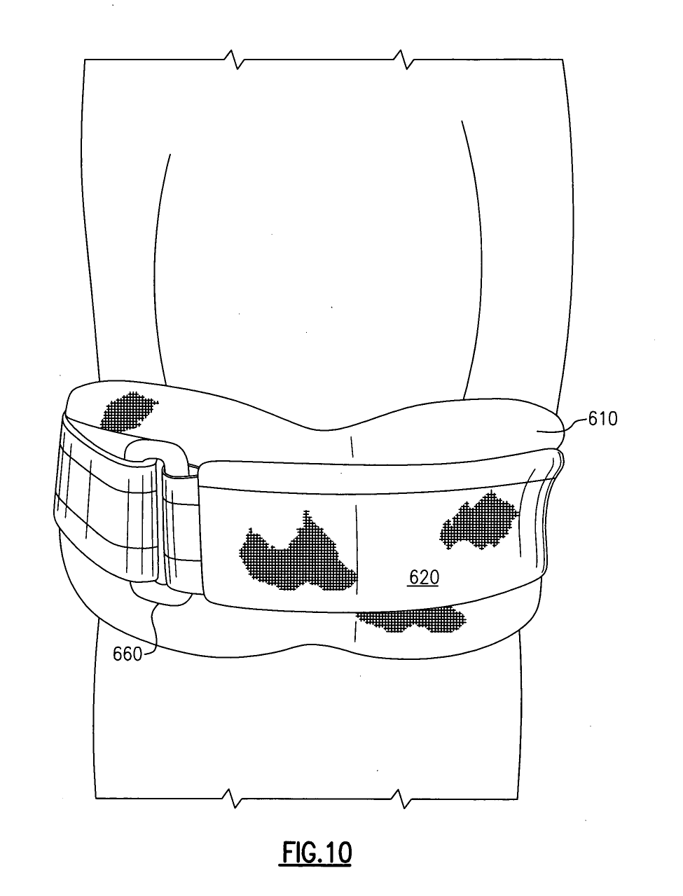

Support buttress 610 can have a variety of shapes; however, as shown in FIGS. 8 and 9, and according to an exemplary embodiment of the present invention, the support buttress has a concave shape resembling a bowtie or hourglass. When the device 100D is worn as shown in FIG. 10, the underside 690 (see FIG. 9) of support buttress 610 is in direct contact with the wearer's leg 200 and the strap 620 is wrapped around the support buttress such that the strap is in direction communication with the strap guide area 685 between the raised portions 680A, 680B of the support buttress.

In accordance with an exemplary embodiment of the present invention, and as best shown in FIG. 9, the inner segment 692 of the underside 690 of support buttress 610 protrudes from the underside by a predetermined height and is formed of an elastomeric material, whereas the outer segment 694 that surrounds the inner segment 692 is formed of a combination of elastomeric material and spacer material. Generally, the elastomeric material that forms the inner segment 692 of the underside 690 of the support buttress 610 has a lower hardness (e.g., 40 Shore 00) than the hardness (e.g., 40 Shore A) of the elastomeric material that forms the outer segment 694.

The orthopedic support devices of the present invention can be formed by one or more techniques known in the art, including, but not limited to, a transfer molding process. Transfer molding is an advantageous manufacturing process in accordance with the present invention because it inexpensively allows a manufacturer to vary one or more of the size, shape, pattern, thickness and location of some or all of the materials that form the end product (i.e., the support device) in order to vary the level/amount and location of compression provided by the support device. Moreover, in further accordance with the present invention, a transfer molding process enables a first material layer to be deposited onto a second material layer or substrate such that some, none or all of the first material layer flows through or encapsulates the second material layer/substrate.

It is understood that although the exemplary embodiments depicted in the figures and described herein pertain either to knee support devices or patella support devices, such devices in accordance with the present invention can be modified without departing from the scope of the present invention so as to be wearable to provide support to other injured body parts, e.g., an elbow, an arm, a leg, an ankle, the back, a shoulder, a wrist, the neck, that can become injured to an extent that the body part requires support or would benefit from being supported while the injured body part heals.

Although the present invention has been described herein with reference to details of currently preferred embodiments, it is not intended that such details be regarded as limiting the scope of the invention, except as and to the extent that they are included in the following claims—that is, the foregoing description of the present invention is merely illustrative, and it should be understood that variations and modifications can be effected without departing from the scope or spirit of the invention as set forth in the following claims. Moreover, any document(s) mentioned herein are incorporated by reference in their entirety, as are any other documents that are referenced within the document(s) mentioned herein.

What is claimed is:

1. An orthopedic support device, comprising:
a support body, wherein the support body is formed of at least two different materials, wherein a first of the at least two different materials is formed directly onto a second of the at least two different materials, and wherein at least the first of the at least two different materials provides targeted compression and a substantially free range of movement to a body part when the support device is worn so as to support the body part, and wherein at least the first of the at least two different materials has at least one of a different modulus and a different hardness than at least the second of the at least two different materials.

2. The orthopedic support device of claim 1, wherein the first of the at least two different materials is an elastomeric material.

3. The orthopedic support device of claim 2, wherein the elastomeric material has a hardness in the range of about Shore 00-30 to about Shore A-50.

4. The orthopedic support device of claim 1, wherein the support body is a sleeve.

5. The orthopedic support device of claim 4, wherein the sleeve has an inner layer and an outer layer, the inner layer being formed of the second of the two different materials and the outer layer being formed of the first of the two different materials.

6. The orthopedic support device of claim 5, wherein the outer layer surrounds only a portion of the inner layer.

7. The orthopedic support device of claim 5, wherein the inner layer is longer than the outer layer.

8. The orthopedic support device of claim 5, wherein the outer layer is formed so as to have a substantially repeating pattern.

9. The orthopedic support device of claim 8, wherein the repeating pattern is a web-like pattern, and wherein a plurality of openings are defined within the outer layer.

10. The orthopedic support device of claim 5, wherein at least one predetermined portion of the outer layer has a thickness greater than the remainder of the outer layer.

11. The orthopedic support device of claim 6, wherein the outer layer is formed as a first shaped buttress.

12. The orthopedic support device of claim 11, wherein a second shaped buttress is formed on the inside of the inner layer.

13. The orthopedic support device of claim 12, wherein the first shaped buttress and the second shaped buttress have substantially identical shapes and dimensions.

14. The orthopedic support device of claim 12, wherein each of the first shaped buttress and the second shaped buttress has a substantially shieldlike shape with an opening defined therein.

15. The orthopedic support device of claim 12, wherein a layer of a third of the at least two different materials is provided between the first shaped buttress and the second shaped buttress.

16. The orthopedic support device of claim 15, wherein the first of the at least two different materials is an elastomeric material, the second of the at least two different materials is a resilient material, and the third of the at least two different materials is a mesh material.

17. The orthopedic support device of claim 12, wherein the first shaped buttress is thicker than the second shaped buttress.

18. The orthopedic support device of claim 12, wherein the first shaped buttress has a lower hardness than the second shaped buttress.

19. The orthopedic support device of claim 1, wherein the support body is a support buttress to which a strap is attached.

20. The orthopedic support device of claim 19, wherein the strap comprises:
a first strap portion formed of hook material; and
a second strap portion formed of a loop material.

21. The orthopedic support device of claim 20, wherein the support buttress comprises:
a first, top side including first and second raised portions formed at least partially of the first of the at least two different materials, wherein a strap guide area formed at least partially of the second of the at least two different materials is defined between the first raised portion and the second raised portion and is configured to accept the strap; and
a second, underside including a raised inner segment formed of the first of the at least two different materials and an outer segment surrounding the inner segment and being formed at least partially of the second of the at least two different materials.

22. The orthopedic support device of claim 20, wherein the first strap portion includes a D-ring.

23. The orthopedic support device of claim 20, wherein the support buttress has a substantially bowtie shape.

24. An orthopedic support sleeve, comprising:
an inner layer formed of a predetermined material; and
an outer layer directly attached to the inner layer and formed of a different predetermined material, wherein the outer layer predetermined material has a different modulus than the inner layer predetermined material, and wherein the outer layer has a predetermined pattern and a plurality of openings defined therein, and wherein the outer layer provides targeted compression and a substantially free range of movement to a body part when the support sleeve is worn so as to support the body part.

25. The orthopedic support sleeve of claim 24, wherein at least one predetermined portion of the outer layer has a thickness greater than the remainder of the outer layer.

26. An orthopedic support sleeve, comprising:
a sleeve body having an inner side and an outer side, wherein the sleeve body is made of a predetermined material and has a sleeve body opening;
a first buttress having a first buttress opening, wherein the first buttress is formed directly onto at least a portion of the inner side of the sleeve body such that at least a portion of the first buttress opening overlaps at least a portion of the sleeve body opening, and wherein the first buttress is made of a predetermined material that is different than the predetermined material of the sleeve body and that has a different hardness than the predetermined material of the support body, and wherein the first buttress provides targeted compression and a substantially free range of movement to a body part when the support sleeve is worn so as to support the body part.

27. The orthopedic support sleeve of claim 26, further comprising:
a second buttress having a second buttress opening, wherein the second buttress is in communication with at least a portion of the outer side of the sleeve body such that at least a portion of the second buttress opening overlaps at least a portion of the sleeve body opening, and wherein the second buttress is made of a predetermined material that is different than the predetermined material of the sleeve body and that has a different hardness than the predetermined material of the sleeve body.

28. An orthopedic support strap device, comprising:
a support buttress, wherein the support buttress comprises:
a first, top side including first and second raised portions formed of a predetermined material, wherein a strap guide is defined between the first and second raised portions and is formed at least partially of a predetermined material that is different than the predetermined material of the first and second raised portion and that has a different elastic hardness than the predetermined material of the first and second raised portions; and
a second, underside; and
a strap portion attached to the top side of the support buttress.

29. The orthopedic support strap of claim 28, wherein the second, underside of the support strap includes a raised inner segment formed at least partially of the same material as the predetermined material of the first and second raised portions and an outer segment surrounding the inner segment and being formed at least partially of the same material as the strap guide, wherein the raised inner segment provides targeted compression and a substantially free range of movement to a body part when the support sleeve is worn so as to support the body part.

30. An orthopedic support device, comprising:
a support body, wherein the support body is formed of at least two different materials, wherein a first of the at least two different materials is formed directly onto a second of the at least two different materials by transfer molding, and wherein at least the first of the at least two different materials provides targeted compression and a substantially free range of movement to a body part when the support device is worn so as to support the body part, and wherein at least the first of the at least two different materials has at least one of a different modulus and a different hardness than at least the second of the at least two different materials.
